(19) United States
(12) Patent Application Publication
(10) Pub. No.: US 2004/0064116 A1
(43) Pub. Date: Apr. 1, 2004

(54) Intravaginal disposable articles having a failure detection system

(76) Inventors: Tarun K. Arora, Edison, NJ (US); Lauren P. Entrekin, Upper Darby, PA (US); Gina J. Marcus, Lawrenceville, NJ (US); H. Michael Moscherosch, Doylestown, PA (US); George G. Quirk IV, Lawrenceville, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/261,320

(22) Filed: Sep. 30, 2002

(51) Int. Cl. A61F 13/15; A61F 13/20

(52) U.S. Cl. 604/361; 604/385.17

(57) ABSTRACT

An intravaginal disposable article, e.g., useful as a sanitary protection device. The intravaginal disposable article has a primary structure and a liquid-sensitive signaling element in liquid communication with the primary structure. The liquid-sensitive signaling element is capable of expanding significantly after exposure of the signaling element to a saturating amount of 0.9 wt-% saline solution.

### FIELD OF THE INVENTION

The present invention relates to intravaginal disposable articles having a liquid-sensitive signaling element that functions as a failure detection device. The failure detection device is capable of signaling the user to change the article as it is approaching capacity or otherwise near failure.

### BACKGROUND OF THE INVENTION

Disposable articles are often used as sanitary protection articles that are designed to contain a particular amount of body exudates such as menstrual fluid. The amount of menstrual fluid absorbed by a napkin, pantiliner or tampon can vary depending on absorbency levels. For example, in the United States, tampon absorbency can range from less than 6 grams (Junior absorbency) grams to 15-18 grams (Ultra absorbency). In other parts of the world, the absorbent capacity may be different. In order to ascertain whether a tampon has reached its absorbent capacity, the tampon must be removed and viewed. Because most women are reluctant to reinsert a partially used tampon, the tampon is usually discarded. In most cases, a user will remove a tampon before it has reached its absorbent capacity in order to prevent fluid from soiling the user's undergarment wherein the absorbent capacity of the tampon is exceeded. Once the absorbent capacity is exceeded, the excess menses can flow unimpeded from the vagina to soil the user's clothing.

Another problem with tampon use is the occasional failure that occurs even when the absorbent capacity is not fully reached. In some cases, the menses will saturate the lower portion of the tampon but the upper portion will remain unsaturated. This may be related to placement of the tampon within the vaginal cavity or to the shape and fit of the tampon.

Passage of menses around an unsaturated tampon is commonly referred to as bypass leakage. As the walls of the vaginal cavity in a normal relaxed state have irregular surfaces and folds, fluid may easily go around the tampon and exit out the body. Additionally, the shape of the tampon may not match the shape of the vagina. In this instance, fluid may travel the gaps and spaces created between the wall and absorbent article and thereby bypass the tampon, i.e., bypass leakage.

A determinative criterion frequently used to gauge tampon replacement is the amount of time elapsed since insertion. This method for changing tampons is not satisfactory for several reasons, e.g., the menstrual flow rate varies throughout the menstruating period and much absorbent capacity of tampons is wasted due to the risk of leakage.

The flow variation throughout the period causes problems as to how long to wear a tampon because a user cannot establish a definite time period for which the absorbent capacity within a tampon is sufficient. A correlation between tampon performance during light flow versus heavy flow is difficult for the user to make, often leaving the user in a quandary as to how long to wear specific tampons during days of heavy flow as contrasted to days of light flow. As a result, the user frequently removes a tampon before the absorbent capacity of the tampon has been reached and wastes much of the product purchased.

One response to this problem has been to make tampons larger and with different materials to obtain higher absorbencies and expansion, often resulting in product claims that a user would not have to change the tampon as often. While the user may have a greater sense of protection, wearing a high absorbency tampon can lead to discomfort and other problems as well. Women will sometimes wear a larger absorbency tampon due to the fear of tampon failure, especially if she is uncertain how often she will be able to access privacy in order to change the tampon. However, if the tampon is unsaturated at the time of removal, there may be excessive drying of the vaginal wall, which may cause discomfort upon the tampon's removal.

One example of such a tampon is disclosed in U.S. Pat. No. 2,254,272 (Crockford). The tampon of Crockford is made from viscose or cellulose sponge material having a substantially stiff or rigid body that softens and expands materially when moistened. Further, Battista et al. (U.S. Pat. No. 4,045,238) discloses saturating regenerated cellulose sponge material so as to uniformly distribute throughout the sponge a film-forming polymer to improve absorbency.

Additionally, if the tampon is unsaturated, the user would still waste a portion of the tampon absorbent capacity as most users are not willing to risk having an accident. Therefore, bigger tampons provide a longer wearing time but do not approach the problem of fully using the absorbent capacity within a tampon. Larger sized tampon can also aid in preventing failure due to bypass leakage.

Kokx et al., U.S. Pat. No. 3,794,024 discloses an indicator in contact with an absorbent body of a catamenial device. The indicator translates wetness into a signal that can be sensed without removal of the catamenial device. Indicating members disclosed in this patent are described as being long enough to allow the terminal end to be viewed by a user. Examples of indicating members include fluid-wicking strings, dyes, chemicals providing temperature changes, and swelling or stiffening agents.

None of the above examples has completely solved the problem of indicating when the an intravaginal absorbent device should be changed in order to prevent leakage. Thus, there is a need for a failure detection system that signals the user to change the sanitary protection article prior to soiling the user's clothing.

### SUMMARY OF THE INVENTION

It is an object of the present invention to provide an intravaginal disposable article having a failure detection system that signals the user to change the article prior to its soiling of a user's clothing.

It is another object of the present invention to provide an intravaginal disposable article having a failure detection system that is located either internally or externally to the user's body.

It is another object of the present invention to provide a method of controlling aqueous vaginal fluids using a disposable article having a failure detection system.

In accordance with one embodiment of the present invention, an intravaginal disposable article having a primary structure and an expandable, liquid-sensitive signaling element in liquid communication with the primary structure is disclosed. The primary structure is sized for insertion into a user's vagina and having a first end capable of being directed toward a source of bodily fluids during use and a second end, opposite the first. The signaling element is separated from the source of bodily fluids during use by the primary structure, and it has an initial volume and capable of expanding to at least about 200% of the initial volume after exposure of the signaling element to a saturating amount of 0.9 wt-% saline solution.

Other aspects of the invention include methods of making such devices and methods of using such devices in the control of aqueous vaginal fluids.

### BRIEF DESCRIPTION OF THE DRAWING

FIG. 1 is an elevational view of one embodiment of the present invention showing a tampon in a compressed, dry state;

FIG. 1A shows an alternative embodiment of an intravaginal device disposed within a user's body;

FIG. 2 shows the tampon of FIG. 1 in a saturated, expanded state;

FIG. 3 is an elevational view of an alternate embodiment of the present invention showing a tampon in a compressed, dry state;

FIG. 4 shows the tampon of FIG. 3 in a saturated, expanded state.

FIG. 5 shows an elevational view of an alternate embodiment of the present invention;

FIG. 6 shows an example of resilient material having a cylindrical shape of material having multiple cuts;

FIG. 7 is a perspective view of an uncompressed resilient material arranged on the withdrawal string for external body placement;

FIG. 8 shows the resilient material of FIG. 7 as compressed;

### DETAILED DESCRIPTION OF THE PREFERRED EMBODIMENTS

Further characteristics and advantages of the invention will become clear from the following detailed description, appended drawings, and non-limiting examples.

As used herein the specification and the claims, the term “liquid communication” and variants thereof relate to the ability of liquid to flow from one element to another.

As used herein the specification and the claims, the term “to saturate” and variants thereof mean to cause to be thoroughly soaked, imbued, or penetrated and include to provide sufficient liquid to dissolve, weaken, or otherwise render a coating insufficient to constrain a resilient material.

As used herein the specification and the claims, the term “resilient” and variants thereof means the capability of a strained body to recover its size and shape after deformation caused especially by bending, compressing, twisting, stretching or any combination thereof. Resilient material can go from a relaxed configuration to a strained configuration to a relaxed configuration at least one and preferably a number of times without losing substantially the ability to recover its original shape. Resilient material in a strained configuration possesses strain energy or potential energy of deformation. It may be desirable that the resilient material be constrained by a coating, sheath or cover, which would weaken upon exposure to fluid.

The present invention is directed to novel intravaginal disposable articles that have a liquid-sensitive, resilient signaling element that functions as a failure detection device. Examples of such articles include, without limitation, internal sanitary protection articles such as tampons, collection cups and other vagina-occluding devices, and pessaries; and the like.

The intravaginal disposable articles include a primary structure and a liquid-sensitive signaling element. Depending upon the desired use of the disposable article, the primary structure may absorb or block the passage of, especially, bodily fluids. If the primary structure is to absorb the bodily fluids, the primary structure will comprise an absorbent structure. If the primary structure is to block the passage of the bodily fluids, it will comprise a structure that occludes the body cavity or collects fluid, such as an inflatable vaginal collection cup as disclosed in Kamen et al., U.S. Pat. No. 6,168,609 B1. The liquid-sensitive signaling element is capable of expanding in size upon contact with sufficient liquid in a manner to provide a detectable signal to the user.

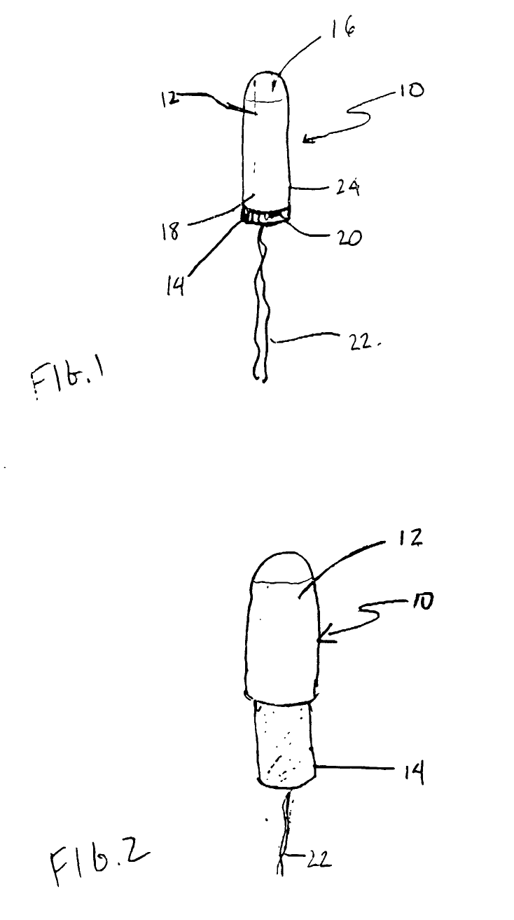

Referring to FIGS. 1 and 2, disposable article 10 is a tampon formed of a primary structure 12 in the form of an absorbent structure, a signaling element 14, a first portion 16, a second portion 18, an edge 20 defining the extreme end of the second portion 18. It may optionally include a withdrawal string 22 and cover (or coating) 24 covering absorbent structure 12. While the signaling element 14 may be associated with any portion of the tampon and may take on many geometries, in the embodiment of FIGS. 1 and 2, it is located adjacent the edge 20 of the tampon and is a disk-like cylinder. While not being bound by any particular theory, it is believed that the lower third of the vagina is more sensitive and would be better able to detect the movement of the signaling element 14 located adjacent the edge 20 of the tampon. Alternately, first portion 16, second portion 18 and signaling element 14 form a substantially unitary absorbent structure 12 to provide direct capillary contact between the absorbent structure 12 and the signaling element 14.

FIG. 1 shows the disposable article 10 in a compressed form, such as when the tampon is removed from its packaging prior to use. Upon insertion into the vaginal cavity, the tampon may begin to absorb bodily fluids, such as menses. After fluid has penetrated into the absorbent structure 12, the absorbent structure 12 expands, e.g., radially. When sufficient absorbed fluids reach the edge 20 of the tampon to be communicated to the signaling element 14, and the signaling element 14 can expand rapidly. The expansion of absorbent structure 12 generally occurs over a relatively long period of time as it absorbs the bodily fluids. This is generally a relatively slow expansion and is not usually detectable by the user. In contrast, signaling element 14 of this embodiment can expand quite rapidly. As signaling element 14 should be in the lower third of the vagina (if the device is properly placed) during the use of the embodiment of FIGS. 1 and 2, the user should be able to feel the expansion of the signaling element 14 along the longitudinal axis of the tampon. FIG. 2 shows this saturated disposable article 10.

In an alternative embodiment shown deployed in a user's vagina (“V”) in FIG. 1A, the absorbent structure 12 is replaced by a non-absorbent structure 12′, e.g., as described in detail in Kamen et al., U.S. Pat. No. 6,168,609 B1, the disclosure of which is herein incorporated by reference. The signaling element 14′ is located in the lower third of the vagina (“I”), while an inflation bulb 15 is located external the body.

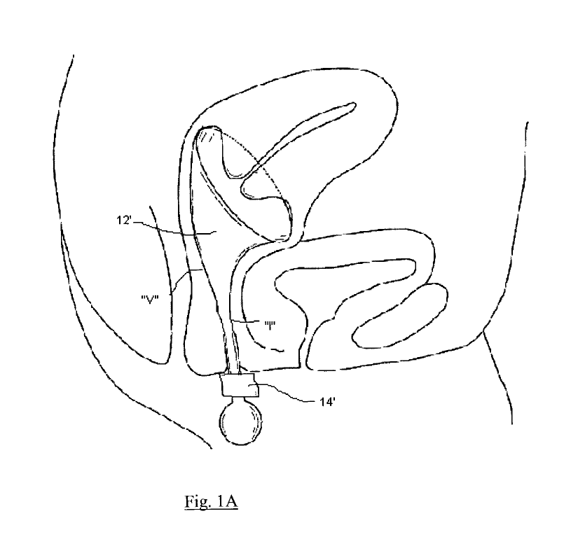

FIGS. 3 and 4 show an alternate embodiment of the invention. In FIG. 3, signaling element 14′ is more of an elongate cylinder than that of the previous embodiment. Again, upon arrival of absorbed fluids at the edge 20 of the tampon, they are communicated to the expandable signaling element 14′. In this embodiment, the signaling element 14′ expands generally uniformly radially outward from the longitudinal axis of the tampon to form the saturated article shown in FIG. 4.

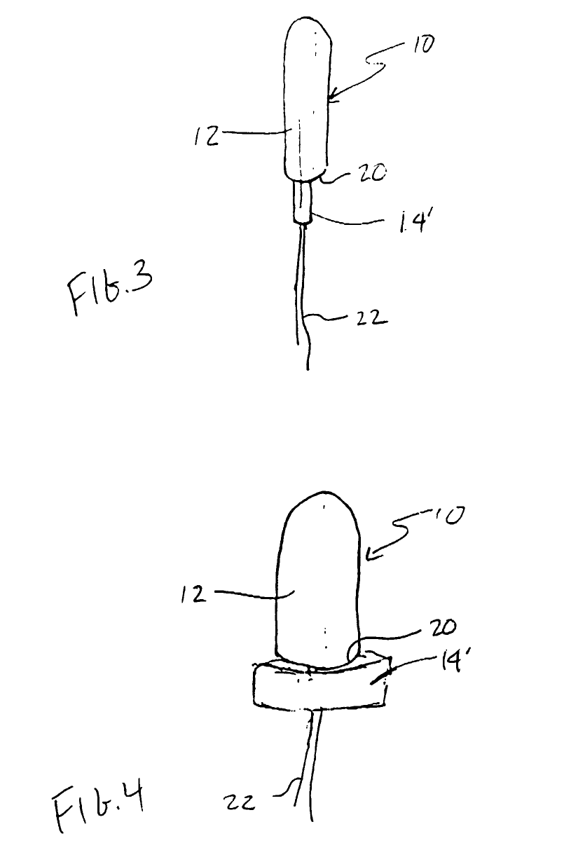

In yet another embodiment, as shown in FIG. 5, signaling element 14″ may be similar in size and shape to that of the embodiment of FIGS. 1 and 2. It is located on the withdrawal string 22. The signaling element 14″ can be securely located at a predetermined position on the string 22, or it may be adjustable. If it is adjustable, the user may place the signaling element 14″ between her labia or elsewhere after the tampon 10 has been inserted. In this embodiment, the signaling element 14″ expands along a single axis perpendicular to the longitudinal axis of the tampon. It is believed that the user can feel the expansion of the signaling element 14″, either by the motion of expansion or by detecting the enlarged form, even with relatively slow expansion.

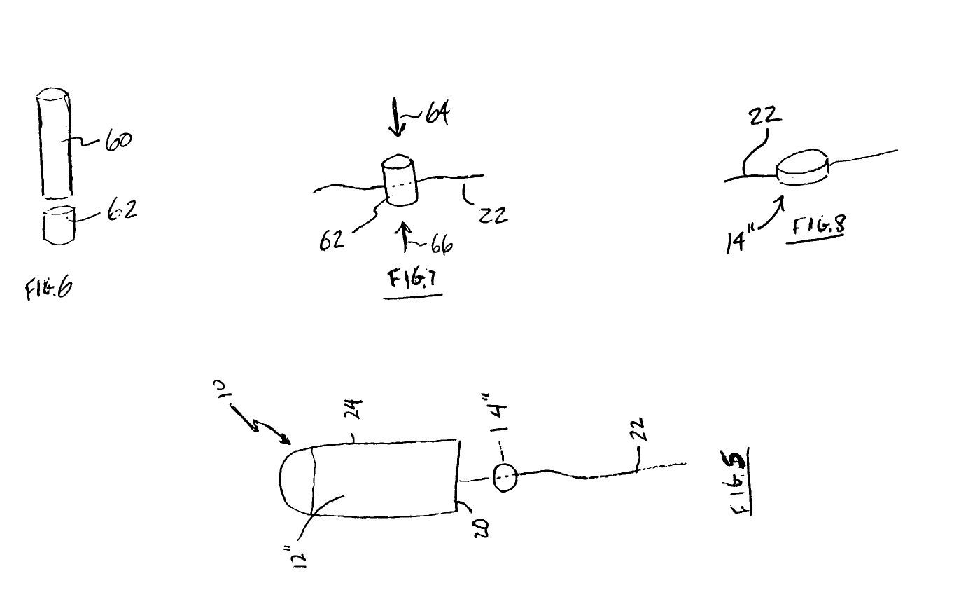

FIGS. 6-8 are illustrative of how the signaling element 14 may be formed. For example, a supply of resilient material in the form of a long cylinder 60 may be cut into slices 62 as seen in FIG. 6. For example, the cylinder may have a diameter that is similar to that of the primary structure with which the signaling element is associated. This may be less than about 20 mm, or even less than 15 mm. The slices may have any desired length, for example, the pre-compression length may be about 25 mm (about 1 inch) or slightly longer or significantly shorter, as short as about 6 mm (about ¼ inch). This slice 62 can be oriented and combined with the primary structure to form the disposable article. For example, the withdrawal string 22 of the embodiment of FIGS. 1 and 2 may be threaded through or attached to a compressed slice to form the signaling element 14. Alternatively, the withdrawal string 22 of the embodiment of FIG. 5 can be threaded through or attached to the slice 62 as shown in FIG. 7. The slice 62 can then be compressed in the direction of arrows 64 and 66 to form the disk-like signaling element 14″ as shown in FIG. 8 having a thickness, e.g., of less than 5 mm. Of course, the resilient material may be compressed at other points in the manufacturing process. It is believed that a user will not greatly notice these unexpanded dimensions, however, it is believed that the user would detect the expanded structure.

If the primary structure 12 is a compressed structure, the order of compression of this structure 12 and the signaling element 14 may occur in any order. The combination string and disc is then used in making the tampon shown in FIG. 8.

Signaling element 14 may comprise any absorbent material that is capable of expansion upon absorption of bodily fluids or any resilient material that is capable of being strained and held in the strained configuration in a dry state and that, upon exposure to liquid (such as blood, menstrual fluid, mucus, saline solution, urine, etc.), reverts to its original, relaxed configuration. The expansion of signaling element 14 may be primarily radially (i.e., generally uniformly outward from a central axis), longitudinally (i.e., lengthwise), sideways (i.e., generally perpendicular along one line perpendicular to a central axis), or any combinations thereof.

Examples of absorbent materials useful in the present invention include but are not limited to hydrogels; modified starches; gelatin; other water soluble polymers that by proper treatment (including but not limited to crosslinking) can be modified to be insoluble while maintaining is affinity to water such as methyl- and hydroxypropyl-methyl-cellulose and derivatives; hydroxyethylcellulose; carboxymethylcellulose; polyvinyl alcohol, polyvinylpyrrolidone, polyacrylic acid and its homologues (a common class of superabsorbents), polyacrylamide, ethylene oxide polymers and polyethylenimine; and the like.

The strained condition may be achieved by distortion in one or more of the following: monoaxially (such as along a longitudinal axis), biaxially (such as along two perpendicular axes), or radially (substantially uniformly inwardly toward a central axis). Preferably, the distortion is a result of compression of the resilient material. Essentially, the resilient material may go from a natural, relaxed configuration into a strained configuration and back to the relaxed configuration (when wet). Examples of such materials include but are not limited to spongy materials, such as of cellulose, polyvinyl alcohol, polyvinyl acetal or formal, polyurethane, or other suitable (usually hydrophilic) foam or sponge material (including open cell sponge); compressed cellulosic fibers; compressed springs made from plastic, metal or any shape memory material; and combinations of different materials to impart variations in composition, density, and porosity; and the like. It is helpful if the materials are biocompatible. For example, a cellulose foam material such as cellulosic sponge may be used. In a natural state, the cellulosic material is spongy and has large pores. In a strained configuration, the cellulosic material is stiffer. Upon relaxation, the cellulosic material “springs” into the spongy state.

The signaling element 14 can be formed into any useful shape. Examples, of the strained configuration of the signaling element 14 are represented in FIGS. 1, 3, 5, and 8. Other shapes will be recognized by the skilled artisan, including without limitation, spherical, ovoid, short disks, long cylinders, cubes, diamonds, etc.

Signaling element 14 has an initial volume prior to use. Upon sufficient penetration of fluid, the signaling element 14 expands to an increased volume. In one form of the present invention, the release of the stored potential energy may cause the liquid-sensitive signaling element to change quickly from an initial volume to a second volume during use. This can be measured in the product by exposing the signaling element to sufficient 0.9 wt-% aqueous saline solution to saturate the signaling element. Preferably, the resilient material in the signaling element changes to its second volume within about 5 seconds, and more preferably within about 2 seconds. The user may then feel this relatively quick movement or motion as a discernible tactile sensation. This sensation would then prompt the user to remove the disposable article and replace it with a new one.

Alternately, it may be the change in volume of the liquid-sensitive signaling element that the user feels. In other words, the signaling element has an initial volume (volume before use or testing) and a second volume achieved after saturation with a 0.9 wt-% aqueous saline solution, and the second volume is larger than the initial volume, generally from about 2 to 20 times (200 to 2000%) its initial volume, and usually from 5 to 15 times (500% to 1500%) its initial volume. The rate at which the resilient material expands is not critical in this alternate form of the invention.

The signaling element may be formed of a single unit, or it may have multiple components. Whichever signaling element used, the change in the signaling element should be subtle enough not to startle the user and yet to elicit an appreciable tactile sensation.

In general, it is necessary that the expansion be significant and of enough volume to be felt by the user. In one embodiment, signaling element 14 is formed from a cellulosic material, such as regenerated cellulosic sponge material. In a natural state, the cellulosic material is spongy and has large pores. In a strained configuration, the cellulosic material is stiffer. Upon relaxation, the cellulosic material “springs” into the spongy state.

The signaling element may have a coating, cover or sheath (“cover”), which will advantageously be dimensioned and configured to contain the signaling element. The cover may be dissolvable or expandable to prevent it from significantly impeding the expansion of the signaling element upon exposure to sufficient moisture. It is understood that in some instances differential compression by the cover may beneficially provide areas of the resilient material having different effective densities. The cover may be used to prevent relaxation or pre-mature expansion until sufficiently weakened by an aqueous or a proteinaceous fluid, or it may be distortable to be capable of expansion in multiple directions and move with the signaling element 20. Additionally, covering or coating the resilient material may be used to isolate the signaling element 14 from the user's body. For example, the cover may prevent any fibers or loose or particulate materials from being left in the vagina or on the body after removal, or it may enclose a spring-like element to protect the user. It has also been found that if the resilient material is, for example, compressed sponge, the resilient material may be stiff and may therefore cause irritation to the vaginal walls until the sponge is saturated. The coating may be any material such as a lubricant, ointment, polymeric film, or the like, which results in a smooth supple outer surface. Useful materials may also be biocompatible. The coating should prevent adherence to body and should ease its removal. Polymeric materials that may be used include, but are not limited to, polyethylene, polypropylene, polyurethane, ethylene vinyl acetate, and silicone; certain polymers can be subjected to a gas plasma or corona discharge treatment, to increase their inherent lubricity. Other materials used may include, without limitation, starch, gelatin, candle wax, polyethylene glycol, polyvinyl alcohol, hydroxypropylcellulose, and polyvinyl propylene, etc. The resilient material may be encased in a capsule formed by a coating or cover.

### EXAMPLE 1

Samples of regenerated cellulosic sponge were cut into approximately 6.5 (L₁) by 6.5 (W₁) by 2.8 (H₁) mm cubes. The samples were individually weighed (w₁) prior to wetting with 0.9 wt-% saline solution. The time required for the saline solution to be absorbed was measured. After wetting, the samples were measured (e.g., L₂) and weighed (w₂). All dimensions were measured in millimeters.

TABLE 1

| Sample | Length | Width | Height | Weight | Absorption |
|---|---|---|---|---|---|
| Number | (L₂-L₁) | (W₂-W₁) | (H₂-H₁) | (w₂-w₁) | time (sec.) |
| 1 | 0.5 | 0.5 | 22.14 | 1.123 | 2 |
| 2 | 0.5 | 0.5 | 22.26 | 1.002 | 2 |
| 3 | 0.0 | 0.5 | 22.26 | 1.124 | 2 |
| 4 | 0.0 | 0.5 | 22.15 | 1.031 | 2 |
| 5 | 0.5 | 0.5 | 22.2 | 0.997 | 2 |
| Average | 0.3 | 0.5 | 22.2 | 1.055 | 2 |

Each sample absorbed approximately 1 gram of fluid within 2 seconds. Absorption of the fluid resulted in an increase of approximately 800% in height.

The foregoing description is intended as illustrative and is not to be taken as limiting. Still other variations are possible without departing from the spirit and scope of this invention and will readily present themselves to one skilled in the art.

What is claimed is:

1. An intravaginal disposable article comprising:
a) a primary structure sized for insertion into a user's vagina and having a first end capable of being directed toward a source of bodily fluids during use and a second end, opposite the first; and
b) a liquid-sensitive signaling element in liquid communication with the primary structure and separated from the source of bodily fluids during use by the primary structure, the signaling element having an initial volume and capable of expanding to at least about 200% of the initial volume after exposure of the signaling element to a saturating amount of 0.9 wt-% saline solution.

2. The intravaginal disposable article of claim 1 wherein the signaling element is spaced from the second end of the primary structure.

3. The intravaginal disposable article of claim 1, wherein the signaling element is spaced a distance from the second end of the primary structure sufficient to be positioned between a user's labia during use.

4. The intravaginal disposable article of claim 1, wherein the signaling element comprises sponge material.

5. The intravaginal disposable article of claim 4, wherein the sponge material comprises regenerated cellulosic sponge.

6. The intravaginal disposable article of claim 4, wherein the sponge material comprises foam.

7. The intravaginal disposable article of claim 1, wherein the signaling element is surrounded with a water soluble material.

8. The intravaginal disposable article of claim 1 wherein a connecting member extends from the second end of the first absorbent device, and the signaling element is movable along the connecting member.

9. The intravaginal disposable article of claim 1 wherein the signaling element is capable of expanding to at least about 500% of the initial volume after exposure of the signaling element to a saturating amount of 0.9 wt-% saline solution

10. The intravaginal disposable article of claim 1, wherein the signaling element comprises absorbent material.

11. The intravaginal disposable article of claim 10, wherein the absorbent material.

12. The intravaginal disposable article of claim 1, wherein the primary structure is absorbent.

13. The intravaginal disposable article of claim 1, wherein the primary structure is non-absorbent.

14. A disposable incontinence device comprising the intravaginal disposable article of claim 1, wherein the liquid-sensitive signaling element is capable of being located adjacent the user's urethra.

15. A method of controlling aqueous vaginal fluids comprising the steps of:
a) inserting a first absorbent device, comprising an elongate primary absorbent structure having a first end directed toward the cervix and a second end opposite the first end, into a vaginal cavity;
b) inserting a signaling element that is in liquid communication with the second end of the primary structure between a set of labia associated with the vaginal cavity, the signaling element having an initial volume and capable of expanding to at least about 200% of the initial volume after exposure of the signaling element to a saturating amount of 0.9 wt-% saline solution;
c) allowing the primary absorbent structure of the first absorbent device to absorb aqueous vaginal fluids;
d) exposing the signaling element to a sufficient amount of aqueous vaginal fluids that have passed the primary absorbent structure to permit the resilient material to expand to at least about 200% of the initial volume;
e) detecting the relatively unstrained configuration of the resilient material; and
f) removing the first disposable article from the vaginal cavity.

16. The method of claim 15 further comprising the step of inserting a second absorbent device into the vaginal cavity after the step of removing the first absorbent device from the vaginal cavity.

17. The method of claim 15 wherein a connecting member extends from the second end of the first absorbent device, and the signaling element is movable along the connecting member.

18. The method of claim 17 wherein the step of inserting the signaling element between the set of labia associated with the vaginal cavity comprises adjusting the distance from the second end of the first absorbent device to the signaling element to correspond to the distance between the second end and the set of labia.

19. The method of claim 15 wherein the signaling element is capable of expanding to at least about 500% of the initial volume after exposure of the signaling element to a saturating amount of 0.9 wt-% saline solution.

20. A method of making a disposable article comprising the steps of:
a) forming a primary structure;
b) manipulating resilient material into a deformed condition;
c) restraining the resilient material in the deformed condition to form a signaling element; and
d) attaching the signaling element to the primary structure.

21. The method of claim 20 wherein the signaling element is attached to the primary structure in the deformed condition.

22. The method of claim 20 wherein the signaling element is attached to the primary structure before the restraining step.

23. The method of claim 20 wherein the signaling element is attached to the primary structure via a connecting member.

24. The method of claim 23 wherein the resilient material is movably attached to the connecting member.
